(19) United States
(12) Patent Application Publication
(10) Pub. No.: US 2024/0082598 A1
(43) Pub. Date: Mar. 14, 2024

(54) OPTICAL SURFACE APPLICATOR WITH FORWARD AND BACKWARD PROJECTION

(71) Applicant: LUMEDA INC., ROCKY HILL, CT (US)

(72) Inventor: Trevor MacDougall, South Dartmouth, MA (US)

(73) Assignee: LUMEDA INC., Rocky Hill, CT (US)

(21) Appl. No.: 18/041,437

(22) PCT Filed: Aug. 12, 2022
(86) PCT No.: PCT/US22/74893

§ 371 Date: Jun. 26, 2023

Related U.S. Application Data
(60) Provisional application No. 63/260,809, filed on Sep. 1, 2021.

(30) Foreign Application Priority Data
Sep. 1, 2021 (US) 63260809

(51) Int. Cl. A61N 5/06

(52) U.S. Cl. CPC A61N 5/062; A61N 2005/063; A61N 2005/0632; A61N 2005/0665

(57) ABSTRACT

A therapy light apparatus and method are disclosed. Also disclosed is an optical assembly comprising a directional light diffuser assembly which comprises a light transmissive hollow elongated tube, an elongated light emitter disposed within the light transmissive hollow elongated tube configured to emit a therapy light, and a reflective coating disposed on a portion of an outer surface of the light transmissive hollow elongated tube configured to reflect the therapy light. Also disclosed is a light delivery apparatus that includes a plurality of a forward directional light diffuser assemblies and rearward a directional light diffuser assemblies.

### CROSS-REFERENCE TO RELATED APPLICATION

This patent application claims priority to U.S. Provisional Patent Application No. 63/260,809, filed on 1 Sep. 2021, and entitled “OPTICAL SURFACE APPLICATOR WITH FORWARD AND BACKWARD PROJECTION”. The disclosure of the prior application is considered part of and is incorporated by reference into this patent application.

### BACKGROUND

### Field of the Disclosure

The present disclosure relates to photodynamic therapy and more specifically to a photodynamic therapy (PDT) light delivery device and method having enhanced capabilities.

### Description of the Related Art

Light therapy can be used for treatment of conditions in multiple ways. For example, some light therapies involve the delivery of a therapeutic light through a fiber optic device placed proximal to or within a target tumor or cancerous tissue.

PDT involves completion of a chemical reaction to produce singlet oxygen to promote cell necrosis. This reaction is dependent on the interplay between its main components: a. type and dose of photosensitizer, b. photosensitizer administration and cellular uptake, and c. total light dose and fluence rate. It is well understood that in practice, PDT efficacy is highly dependent upon proper light dose and fluence rate i.e. dosimetry.

PDT light delivery can use a Freiburg flap or H.A.M. as an optical surface applicator (OSA) with cylindrical light diffusers disposed therein. Current OSA's only have the ability to project treatment light in one planar direction (forward). What is needed is a device and method for that can project therapy light in a forward and rearward direction.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, an optical assembly may include a directional light diffuser assembly having a light transmissive hollow elongated tube configured to receive a light emitter, and a reflective coating disposed on a portion of the light transmissive hollow elongated tube configured to reflect a therapy light. Other embodiments of this aspect include corresponding computer systems, apparatus, and computer programs recorded on one or more computer storage devices, each configured to perform the actions of the methods.

Implementations may include one or more of the following features. The optical assembly may include an elongated light emitter configured to emit the therapy light disposed within the light transmissive hollow elongated tube. The optical assembly where the reflective coating is disposed on a portion of an outer surface of the light transmissive hollow elongated tube. The optical assembly where the directional light diffuser assembly may include a forward directional light diffuser assembly where the reflective coating is disposed on a top outer surface of the light transmissive hollow elongated tube, and a rearward directional light diffuser assembly where the reflective coating is disposed on a bottom outer surface of the light transmissive hollow elongated tube. The optical assembly may include a light transmissive optical surface applicator having a plurality of elongated channels disposed therein, a plurality of the forward directional light diffuser assemblies disposed in a first portion of the plurality of elongated channels, and a plurality of the rearward directional light diffuser assemblies disposed in a second portion of the plurality of elongated channels. The optical assembly where the elongated light emitter is a cylindrical light diffuser. The optical assembly where the reflective coating is may include of a gold based material.

In one general aspect, optical light delivery system may include a therapy light source configured to produce a therapy light. The optical light delivery system may also include a forward directional light diffuser assembly having a light transmissive hollow elongated tube, an elongated light emitter disposed within the light transmissive hollow elongated tube, and a reflective coating disposed on a top outer surface of the light transmissive hollow elongated tube. The system may furthermore include a rearward directional light diffuser assembly having a light transmissive hollow elongated tube, an elongated light emitter disposed within the light transmissive hollow elongated tube, a reflective coating disposed on a bottom outer surface of the light transmissive hollow elongated tube configured to reflect the therapy light. The system may in addition include a light transmissive optical surface applicator having a plurality of elongated channels disposed therein. The system may moreover include a plurality of the forward directional light diffuser assemblies disposed in a first portion of the plurality of elongated channels. The system may also include a plurality of the rearward directional light diffuser assemblies disposed in a second portion of the plurality of elongated channels. The system may furthermore include a computer processor electrically coupled to the therapy light source. The system may in addition include a light source controller electrically coupled to the computer processor and optically coupled to the plurality of the forward directional light diffuser assemblies and optically coupled to the plurality of the rearward directional light diffuser assemblies. Other embodiments of this aspect include corresponding computer systems, apparatus, and computer programs recorded on one or more computer storage devices, each configured to perform the actions of the methods.

Implementations may include one or more of the following features. An optical light delivery system where the light source controller is configured to selectively deliver the therapy light to the plurality of the forward directional light diffuser assemblies and to the plurality of the rearward directional light diffuser assemblies. The optical light delivery system may include an optical tether to optically couple the light source controller to the plurality of the forward directional light diffuser assemblies and to the plurality of the rearward directional light diffuser assemblies. The optical light delivery system may include an instrument, where the instrument may include the therapy light source, a spectrum analyzer, the computer processor and a display. The optical light delivery system may include a digital file of a position of an abnormal tissue area, and where the instrument selectively delivers the therapy light to any of the plurality of the forward directional light diffuser assemblies and the plurality of the rearward directional light diffuser assemblies to produce an irradiance pattern that closely matches the abnormal tissue area. The optical light delivery system where the instrument produces the irradiance pattern to produce a total light dose. Implementations of the described techniques may include hardware, a method or process, or a computer tangible medium.

In one general aspect, a method may include providing a light transmissive hollow elongated tube configured to receive a light emitter. The method may also include disposing a reflective coating on a portion of the light transmissive hollow elongated tube configured to reflect a therapy light.

Implementations may include one or more of the following features. The method may include disposing an elongated light emitter disposed within the light transmissive hollow elongated tube. The method may include disposing the reflective coating on a portion of an outer surface of the light transmissive hollow elongated tube. The method where the optical assembly may include providing a forward directional light diffuser assembly by disposing the reflective coating is on a top outer surface of the light transmissive hollow elongated tube, and providing a rearward directional light diffuser assembly by disposing the reflective coating on a bottom outer surface of the light transmissive hollow elongated tube. The method may include providing a light transmissive optical surface applicator having a plurality of elongated channels disposed therein, disposing a plurality of the forward directional light diffuser assemblies in a first portion of the plurality of elongated channels, and disposing a plurality of the rearward directional light diffuser assemblies in a second portion of the plurality of elongated channels. The method where the elongated light emitter is a cylindrical light diffuser. The method where the reflective coating is may include of a gold based material.

In one general aspect, a method may include providing a therapy light source. The method may also include providing a forward directional light diffuser assembly having providing a light transmissive hollow elongated tube, disposing an elongated light emitter within the light transmissive hollow elongated tube, and disposing a reflective coating on a top outer surface of the light transmissive hollow elongated tube. The method may furthermore include providing a rearward directional light diffuser assembly having providing a light transmissive hollow elongated tube, disposing an elongated light emitter within the light transmissive hollow elongated tube, disposing a reflective coating disposed on a bottom outer surface of the light transmissive hollow elongated tube configured to reflect the therapy light. The method may in addition include providing a light transmissive optical surface applicator having a plurality of elongated channels disposed therein. The method may moreover include disposing a plurality of the forward directional light diffuser assemblies in a first portion of the plurality of elongated channels. The method may also include disposing a plurality of the rearward directional light diffuser assemblies in a second portion of the plurality of elongated channels. The method may furthermore include electrically coupling a computer processor to the therapy light source. The method may in addition include electrically coupling a light source controller to the computer processor, and optically coupling the light source controller to the plurality of the forward directional light diffuser assemblies and to the plurality of the rearward directional light diffuser assemblies. The method may moreover include delivering the therapy light to any of the plurality of the forward directional light diffuser assemblies and the plurality of the rearward directional light diffuser assemblies. Other embodiments of this aspect include corresponding computer systems, apparatus, and computer programs recorded on one or more computer storage devices, each configured to perform the actions of the methods.

Implementations may include one or more of the following features. The method may include selectively delivering the therapy light to the plurality of the forward directional light diffuser assemblies and to the plurality of the rearward directional light diffuser assemblies. The method may include coupling the light source controller to the plurality of the forward directional light diffuser assemblies and to the plurality of the rearward directional light diffuser assemblies with an optical tether. The method may include providing an instrument and disposing the therapy light source, a spectrum analyzer, the computer processor and a display in the instrument. The method may include obtaining a digital file of a position of an abnormal tissue area, positioning the light transmissive optical surface applicator at the position of the abnormal tissue area, and selectively delivering, using the instrument, the therapy light to any of the plurality of the forward directional light diffuser assemblies and the plurality of the rearward directional light diffuser assemblies, and producing an irradiance pattern that closely matches the abnormal tissue area. The method may include producing a total light dose. The method may include administering a photosensitizing drug to the patient, performing a primary treatment to remove a gross portion of an abnormal tissue in the abnormal tissue area, and performing an adjuvant therapy to the abnormal tissue area using the therapy light. Implementations of the described techniques may include hardware, a method or process, or a computer tangible medium. The method may include administering a photosensitizing drug to the patient, performing a primary treatment to remove a gross portion of an abnormal tissue in the abnormal tissue area, and performing photodynamic therapy to the abnormal tissue area using the therapy light. Method where the photodynamic therapy can include any of a necrotic therapy, an apoptotic therapy, a vascular therapy and an immunogenicity therapy.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only typical implementations of this disclosure and are therefore not to be considered limiting of its scope, for the disclosure may admit to other equally effective implementations.

FIG. 1 is a cross sectional view of a directional light diffuser assembly in accordance with the present disclosure;

FIG. 2 is a side view of a forward a directional light diffuser assembly in accordance with the present disclosure;

Figure is a side view of a rearward a directional light diffuser assembly in accordance with the present disclosure;

FIG. 4 is a schematic representation of optical surface applicator in accordance with the present disclosure;

FIG. 5 is a schematic representation of optical surface applicator in accordance with the present disclosure;

FIG. 6 shows a top view schematic representation of an implementation of optical surface applicator in accordance with the present disclosure;

FIG. 7 is a section view of optical surface applicator assembly taken along cut line 7-7 in FIG. 6 in accordance with the present disclosure; and

FIG. 8 is a schematic view of one implementation of an optical light delivery system in accordance with the present disclosure.

### DETAILED DESCRIPTION

In the following detailed description of the implementations, reference is made to the accompanying drawings, which form a part hereof, and within which are shown by way of illustration specific implementations by which the examples described herein may be practiced. It is to be understood that other implementations may be utilized and structural changes may be made without departing from the scope of the disclosure.

Implementations of the present disclosure provide a means of delivery light therapy during a PDT procedure using an OSA that can be switched while in position to project therapy light from a forward direction to a reverse direction (backward).

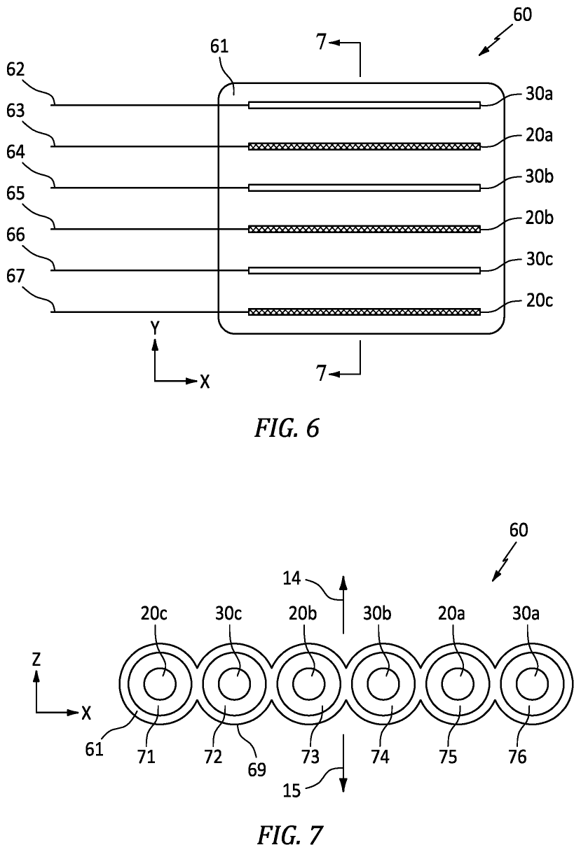

Implementations of the present disclosure include a tube half coated with a gold reflecting layer surrounding a cylindrical light diffuser (CLD) wherein the gold layer directs the light radiating from the CLD in a particular direction (i.e. in a direction away from the gold coating). Other implementations include an array of CLD's positioned within a respective coated tubes arranged in an OSA (FIGS. 6-8) so that they alternate in a way such that some CLD's of the array project therapy light in a first (forward) direction and others project therapy light in a second (reverse or backward) direction. As will be disclosed in more detail herein after further implementations include means by which each of the CLD's in the array can be selected electronically individually such that they could be programmed in real time to illuminate and to project therapy light in the forward direction, the backward direction or in both directions.

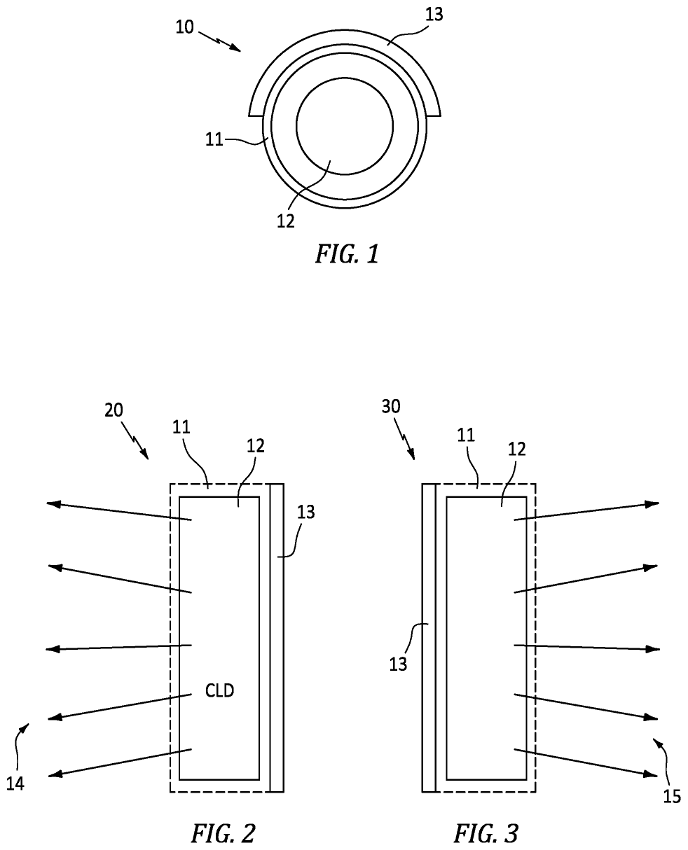

Referring to FIG. 1, there is shown a cross sectional view of a directional light diffuser assembly 10 comprised of tubing 11 and cylindrical light diffuser 12 disposed within the tubing. Tubing 11 further includes reflective layer 13 positioned on an outer surface of the tubing. Reflective layer 13 can comprise any material capable of reflecting therapy light away from the reflective coating and back into the inner diameter of tubing 11 and in some implementations comprises a gold based material. Reflective layer 13 can be applied to a portion of the outer diameter of tubing 11 using any suitable process including sputtering, plasma spray, pressure sensitive adhesive layers and the like. Tubing 11 can be comprised of a glass material or other suitable material having light transmissibility qualities configured to allow therapy light to escape and which material can further withstand the temperatures of the coating process of reflective layer 13.

Referring next to FIG. 2, there is shown a forward directional light diffuser assembly 20 taken along a cut line 2-2 in FIG. 1 and inverted. Forward directional light diffuser assembly 20 comprises tubing 11 with cylindrical light diffuser 12 disposed therein and reflective coating 13 disposed on an outer diameter thereof similar to that disclosed herein above with reference to directional light diffuser assembly 10. In this particular implementation, reflective coating 13 is positioned on a bottom outer surface of tubing 11 such that therapy light emitted from cylindrical light diffuser 12 that becomes incident on the reflective coating is reflected back into the tubing. With reflective coating 13 positioned on the bottom side of tubing 11 all the therapy light emitted by cylindrical light diffuser 12 is transmitted through a top side of forward directional light diffuser assembly 20 in a forward (or first) direction indicated by arrows 14. As will be disclosed in further detail with reference to FIGS. 6-8, cylindrical light diffuser 12 can be optically coupled to an optical fiber in communication with a light source, a light source controller and a computer processor for controllably delivering therapy light to forward directional light diffuser assembly 20. Referring now to FIG. 3, there is shown a rearward directional light diffuser assembly 30 taken along a cut line 2-2 in FIG. 1. Rearward directional light diffuser assembly 30 comprises tubing 11 with cylindrical light diffuser 12 disposed therein and reflective coating 13 disposed on an outer diameter thereof similar to that disclosed herein above with reference to directional light diffuser assembly 10. In this particular implementation reflective coating 13 is positioned on a top side of tubing 11 such that therapy light emitted from cylindrical light diffuser 12 that becomes incident on the reflective coating is reflected back into the tubing. With reflective coating 13 positioned on the top outer surface of tubing 11 all the therapy light emitted by cylindrical light diffuser 12 is transmitted through a bottom side of forward directional light diffuser assembly 30 in a rearward (or second) direction indicated by arrows 15. Similar to forward directional light diffuser assembly 20, and as will be disclosed in further detail herein after, and as part of an optical light delivery system, cylindrical light diffuser 12 can be optically coupled to an optical fiber in communication with a light source, a light source controller and a computer processor for controllably delivering therapy light to forward directional light diffuser assembly 30.

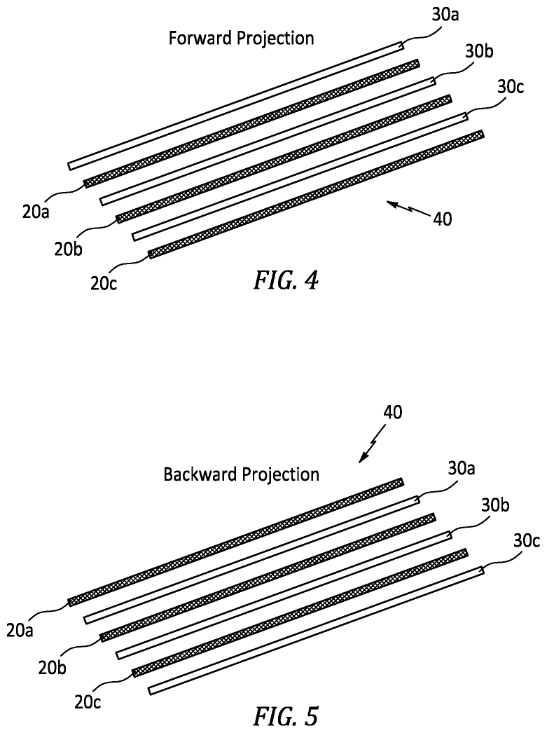

Referring next to FIG. 4, there is shown a topside layout of an optical surface applicator 40 configured to project therapy light in a forward direction (14 in FIG. 2). Optical surface applicator 40 can include elongated channels disposed therein and is comprised of forward directional light diffuser assemblies 20a, 20b, 20c and rearward directional light diffuser assemblies 30a, 30b, 30c variously disposed within the elongated channels. In this particular implementation forward directional light diffuser assemblies 20a, 20b, 20c are configured to project therapy light from a light source in the forward direction. Now with reference to FIG. 5, there is shown a bottom side layout of optical surface applicator 40 configured to project therapy light in a forward direction (1 in FIG. 3). Optical surface applicator 40 depicted in FIG. 5 has been turned upside down from that shown in FIG. 4 by flipping it from left to right. In this particular implementation forward directional light diffuser assemblies 30a, 30b, 30c are configured to project therapy light from a light source in the forward direction. Although not shown in the figures forward directional light diffuser assemblies 20a, 20b, 20c and rearward directional light diffuser assemblies 30a, 30b, 30c can be controlled in an on/off state by a light controller optically coupled to a therapy light source such that any combination of the forward directional light diffuser assemblies and rearward directional light diffuser assemblies can be energized in accordance with a therapy plan.

Referring next to FIGS. 6-7, there is shown an optical surface applicator assembly 60 with an implementation of optical surface applicator 61. FIG. 6 shows a top view of an implementation of optical surface applicator 61 wherein the optical surface applicator generally defines a plane in the X-Y space. optical surface applicator 61 includes forward directional light diffuser assemblies 20a, 20b, 20c optically coupled to optical fibers 63, 65, 67 respectively as well as rearward directional light diffuser assemblies 30a, 30b, 30c optically coupled to optical fibers 62, 64, 66 respectively. FIG. 7 is a section view of optical surface applicator assembly 60 taken along cut line 7-7 in FIG. 6 through optical surface applicator 61. Optical surface applicator 61 comprises a light transmissive optical surface applicator comprised of light transmissible material having a forward side 68 and a backward side 69 and elongated channels 71-76 distributed along the X direction of the optical surface applicator. The forward directional light diffuser assemblies 20a, 20b, 20c and the rearward directional light diffuser assemblies 30a, 30b, 30c are disposed in a respective elongated channel 71-76. As disclosed herein above, forward directional light diffuser assemblies 20a, 20b, 20c include a reflective layer 13 disposed on an outer diameter of tube 11 to reflect light away from backward side 68 such that the therapy light projected from the forward directional light diffuser assemblies is directed in the forward direction 14. Similarly, rearward directional light diffuser assemblies 30a, 30b, 30c include a reflective layer 13 disposed on an outer diameter of tube 11 to reflect light away from forward side 69 such that the therapy light projected from the rearward directional light diffuser assemblies is directed in the rearward direction 15.

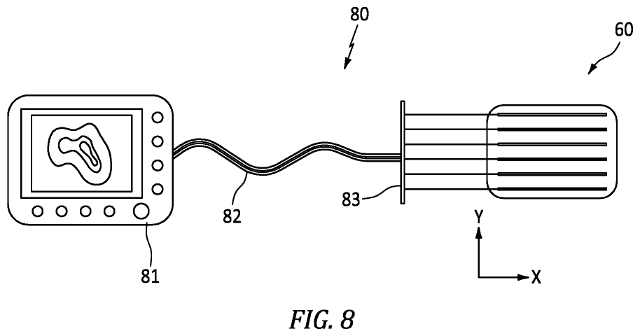

Referring now to FIG. 8, there is shown a schematic view of one implementation of an optical light delivery system 80 employing optical surface applicator 60 for use in a photodynamic therapy procedure as will be disclosed in more detail herein after. The optical light delivery system 80 is comprised of a number of components including an instrument 81, optical surface applicator 60 and an optical tether 82 optically coupling the instrument to the integrated optical applicator via optical connector 83 and optical fibers 62-67 (FIG. 6). Instrument 81 includes a light source, a spectrum analyzer, a computer processor and an optional display all of which may be electrically coupled. In optical light delivery system 80, therapy light is provided by the source in instrument 81 and selectively delivered to the tissue of a patient (not shown) by forward directional light diffuser assemblies 20a, 20b, 20c and rearward directional light diffuser assemblies 30a, 30b, 30c. Optical tether 82 includes individual optical fibers sufficient in number to optically couple to a single one of the optical fibers 62-67 and can be connected by an optical connector 83. In operation, a medical professional can obtain a digital file (x-ray, magnetic resonance imaging (MRI), computer aided tomography (CAT) scan, etc.) of a position of an abnormal tissue area to be treated using a PDT procedure. The digital file can contain at least X and Y coordinates of the area relative to a position in space. A patient is administered a photosensitizing drug and is prepared for a surgical treatment wherein a gross portion of the abnormal tissue is typically surgically removed as a primary treatment. The PDT procedure can be used as adjuvant therapy to kill of any residual amounts of abnormal tissue left behind. In addition, the PDT treatment can be used to enhance a response to immuno-therapy treatments. It should be appreciated that the therapy type can be selected from the group that may include necrotic, apoptotic, vascular and immunogenicity. An optical surface applicator 60 is prepared with forward directional light diffuser assemblies 20a, 20b, 20c and rearward directional light diffuser assemblies 30a, 30b, 30c as disclosed herein above to produce a target irradiance pattern that closely matches the abnormal tissue area to be treated using a PDT procedure. A treatment plan is devised by medical personnel based on, among other things, the type of therapy, the source, the fluence of optical surface applicator 60, a dosage therapy light amount and the specific photosensitizing drug, wherein the treatment plan is directed at treating the abnormal tissue in the target area. The integrated optical applicator is positioned proximal the abnormal tissue area to be treated using a PDT procedure with forward side 68 and/or backward side 69 positioned in direct contact with the tissue to be treated. The optical surface applicator 60 applicator is positioned using the X and Y coordinates of the digital file and a digital file of the target irradiance pattern. Instrument 81 is powered on and therapy light is selectively delivered to forward directional light diffuser assemblies 20a, 20b, 20c and/or rearward directional light diffuser assemblies 30a, 30b, 30c via optical tether 82, connector 83 and optical fibers 62-67. The therapy light is applied to the target area through forward side 68 and/or backward side 69 of optical surface applicator 60. The procedure is continued until the dosage therapy light amount calculated by the computer processor of instrument 81 for each of selected forward directional light diffuser assemblies 20a, 20b, 20c and/or rearward directional light diffuser assemblies 30a, 30b, 30c is substantially equal to the treatment plan. In some implementations, the treatment plan can be updated based on the original treatment plan and calculations made by the computer processor.

All of the implementations disclosed and claimed herein can be made and executed without undue experimentation in light of the present disclosure. While the apparatus and methods of this disclosure have been described in terms of preferred implementations, it will be apparent to those of skill in the art that variations may be applied to the methods and in the steps or in the sequence of steps of the method described herein without departing from the concept, spirit and scope of the disclosure. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the disclosure.

Although the disclosure(s) is/are described herein with reference to specific implementations, various modifications and changes can be made without departing from the scope of the present disclosure, as presently set forth in the claims below. Accordingly, the specification and figures are to be regarded in an illustrative rather than a restrictive sense, and all such modifications are intended to be included within the scope of the present disclosure. Any benefits, advantages, or solutions to problems that are described herein with regard to specific implementations are not intended to be construed as a critical, required, or essential feature or element of any or all the claims.

Unless stated otherwise, terms such as “first” and “second” are used to arbitrarily distinguish between the elements such terms describe. Similarly, terms such as forward and backward are used to distinguish from opposite directions of travel. Thus, these terms are not necessarily intended to indicate temporal, absolute directional or other prioritization of such elements. The terms “coupled” or “operably coupled” are defined as connected, although not necessarily directly, and not necessarily mechanically. The terms “a” and “an” are defined as one or more unless stated other The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements. Similarly, a method or process that “comprises,” “has,” “includes” or “contains” one or more operations possesses those one or more operations but is not limited to possessing only those one or more operations.

While the foregoing is directed to implementations of the present disclosure, other and further implementations of the disclosure may be devised without departing from the basic scope thereof, and the scope thereof is determined by the claims that follow.

1. An optical assembly comprising:
a directional light diffuser assembly comprising:
a light transmissive hollow elongated tube configured to receive a light emitter;
a reflective coating disposed on a portion of the light transmissive hollow elongated tube configured to reflect a therapy light;
wherein the reflective coating is disposed on a portion of an outer surface of the light transmissive hollow elongated tube; and
wherein the directional light diffuser assembly comprises:
a forward directional light diffuser assembly wherein the reflective coating is disposed on a top outer surface of the light transmissive hollow elongated tube; and
a rearward directional light diffuser assembly wherein the reflective coating is disposed on a bottom outer surface of the light transmissive hollow elongated tube.

2. The optical assembly of claim 1 further comprising an elongated light emitter configured to emit the therapy light disposed within the light transmissive hollow elongated tube.

3. (canceled)

4. The optical assembly of claim 1 wherein the reflective coating is comprised of a gold based material.

5. The optical assembly of claim 2 wherein the elongated light emitter is a cylindrical light diffuser.

6. (canceled)

7. The optical assembly of claim 1 further comprising:
a light transmissive optical surface applicator having a plurality of elongated channels disposed therein;
a plurality of the forward directional light diffuser assemblies disposed in a first portion of the plurality of elongated channels; and
a plurality of the rearward directional light diffuser assemblies disposed in a second portion of the plurality of elongated channels.

8. An optical light delivery system comprising:
a therapy light source configured to produce a therapy light;
a forward directional light diffuser assembly comprising:
a light transmissive hollow elongated tube;
an elongated light emitter disposed within the light transmissive hollow elongated tube; and
a reflective coating disposed on a top outer surface of the light transmissive hollow elongated tube;
a rearward directional light diffuser assembly comprising:
a light transmissive hollow elongated tube;
an elongated light emitter disposed within the light transmissive hollow elongated tube;
a reflective coating disposed on a bottom outer surface of the light transmissive hollow elongated tube configured to reflect the therapy light;
a light transmissive optical surface applicator having a plurality of elongated channels disposed therein;
a plurality of the forward directional light diffuser assemblies disposed in a first portion of the plurality of elongated channels;
a plurality of the rearward directional light diffuser assemblies disposed in a second portion of the plurality of elongated channels;
a computer processor electrically coupled to the therapy light source; and
a light source controller electrically coupled to the computer processor and optically coupled to the plurality of the forward directional light diffuser assemblies and optically coupled to the plurality of the rearward directional light diffuser assemblies.

9. The optical light delivery system of claim 8 wherein the light source controller is configured to selectively deliver the therapy light to the plurality of the forward directional light diffuser assemblies and to the plurality of the rearward directional light diffuser assemblies.

10. The optical light delivery system of claim 9 further comprising an optical tether to optically couple the light source controller to the plurality of the forward directional light diffuser assemblies and to the plurality of the rearward directional light diffuser assemblies.

11. The optical light delivery system of claim 10 further comprising an instrument, wherein the instrument comprises the therapy light source, a spectrum analyzer, the computer processor and a display.

12. The optical light delivery system of claim 11 further comprising a digital file of a position of an abnormal tissue area; and
wherein the instrument selectively delivers the therapy light to any of the plurality of the forward directional light diffuser assemblies and the plurality of the rearward directional light diffuser assemblies to produce an irradiance pattern that closely matches the abnormal tissue area.

13. The optical light delivery system of claim 12 wherein the instrument produces the irradiance pattern to produce a total light dose.

14. A method of producing an optical assembly comprising:
providing a light transmissive hollow elongated tube configured to receive a light emitter;
disposing a reflective coating on a portion of the light transmissive hollow elongated tube configured to reflect a therapy light;
disposing an elongated light emitter disposed within the light transmissive hollow elongated tube;
disposing the reflective coating on a portion of an outer surface of the light transmissive hollow elongated tube;
providing a forward directional light diffuser assembly by disposing the reflective coating is on a top outer surface of the light transmissive hollow elongated tube; and
providing a rearward directional light diffuser assembly by disposing the reflective coating on a bottom outer surface of the light transmissive hollow elongated tube.

15. (canceled)

16. (canceled)

17. The method of producing an optical assembly of claim 14 wherein the reflective coating is comprised of a gold based material.

18. The method of producing an optical assembly of claim 15 wherein the elongated light emitter is a cylindrical light diffuser.

19. (canceled)

20. The method of producing an optical assembly of claim 14 further comprising:
providing a light transmissive optical surface applicator having a plurality of elongated channels disposed therein;
disposing a plurality of the forward directional light diffuser assemblies in a first portion of the plurality of elongated channels; and
disposing a plurality of the rearward directional light diffuser assemblies in a second portion of the plurality of elongated channels.

21. A method of delivering a therapy light to a patient comprising:
providing a therapy light source;
providing a forward directional light diffuser assembly comprising:
providing a light transmissive hollow elongated tube;
disposing an elongated light emitter within the light transmissive hollow elongated tube; and
disposing a reflective coating on a top outer surface of the light transmissive hollow elongated tube;
providing a rearward directional light diffuser assembly comprising:
providing a light transmissive hollow elongated tube;
disposing an elongated light emitter within the light transmissive hollow elongated tube;
disposing a reflective coating disposed on a bottom outer surface of the light transmissive hollow elongated tube configured to reflect the therapy light;
providing a light transmissive optical surface applicator having a plurality of elongated channels disposed therein;
disposing a plurality of the forward directional light diffuser assemblies in a first portion of the plurality of elongated channels;
disposing a plurality of the rearward directional light diffuser assemblies in a second portion of the plurality of elongated channels;
electrically coupling a computer processor to the therapy light source; and
electrically coupling a light source controller to the computer processor; and
optically coupling the light source controller to the plurality of the forward directional light diffuser assemblies and to the plurality of the rearward directional light diffuser assemblies; and
delivering the therapy light to any of the plurality of the forward directional light diffuser assemblies and the plurality of the rearward directional light diffuser assemblies.

22. The method of delivering a therapy light to a patient of claim 21 further comprising selectively delivering the therapy light to the plurality of the forward directional light diffuser assemblies and to the plurality of the rearward directional light diffuser assemblies.

23. The method of delivering a therapy light to a patient of claim 22 further comprising coupling the light source controller to the plurality of the forward directional light diffuser assemblies and to the plurality of the rearward directional light diffuser assemblies with an optical tether.

24. The method of delivering a therapy light to a patient of claim 23 further comprising providing an instrument and disposing the therapy light source, a spectrum analyzer, the computer processor and a display in the instrument.

25. The method of delivering a therapy light to a patient of claim 24 further comprising obtaining a digital file of a position of an abnormal tissue area;
positioning the light transmissive optical surface applicator at the position of the abnormal tissue area; and
selectively delivering, using the instrument, the therapy light to any of the plurality of the forward directional light diffuser assemblies and the plurality of the rearward directional light diffuser assemblies; and
producing an irradiance pattern that closely matches the abnormal tissue area.

26. The method of delivering a therapy light to a patient of claim 25 further comprising producing a total light dose.

27. The method of delivering a therapy light to a patient of claim 26 further comprising:
administering a photosensitizing drug to the patient;
performing a primary treatment to remove a gross portion of an abnormal tissue in the abnormal tissue area; and
performing photodynamic therapy to the abnormal tissue area using the therapy light.

28. The method of delivering a therapy light to a patient of claim 27 wherein the photodynamic therapy can include any of a necrotic therapy, an apoptotic therapy, a vascular therapy and an immunogenicity therapy.
